(19) United States
(12) Patent Application Publication
(10) Pub. No.: US 2018/0127710 A1
(43) Pub. Date: May 10, 2018

(54) STEM CELL CARRIER AND METHOD FOR BONE REGENERATION WITH 3D CUSTOMIZED CAD/CAM USING THE CARRIER

(71) Applicant: EZEKIEL CO., LTD, Chungcheongnam-do (KR)

(72) Inventor: Jaejoon LEE, Chungcheongnam-do (KR)

(21) Appl. No.: 14/556,006

(22) Filed: Nov. 28, 2014

(51) Int. Cl. C12N 5/071; A61B 5/00; A61B 5/055; A61B 6/03; A61F 2/28

(52) U.S. Cl. CPC C12N 5/0602; A61B 5/0035; A61B 5/055; A61B 6/032; A61F 2002/2835; A61F 2/28; C12N 2513/00; C12N 2533/18; A61B 5/4836

(57) ABSTRACT

A stem cell carrier includes a plurality of single units. The plurality of single units are formed three-dimensionally interconnected to each other to form a cubic structure. The plurality of single units are made of a porous material. Each single unit has three-dimensionally formed through holes crossing each other, and each through hole has 400 to 500 micrometers in diameter. The through holes of the plurality of single units are interconnected to each other to form a plurality of channels which are formed from one side of the cubic structure to the other side.

### BACKGROUND OF THE INVENTION

### 1. Field of the Invention

The present invention relates to a stem cell carrier and, more particularly, to a stem cell carrier produced from a biocompatible material and a method for bone regeneration using 3D customized computer-aided design and computer-aided manufacturing (CAD/CAM) without using a graft that is simply filled in a defect in bone tissue.

### 2. Description of the Related Art

A stem cell is a primitive cell and is also referred to as a pluripotent cell or progenitor cell that can grow into any organ. Stem cells include embryonic stem cells and adult stem cells.

The present invention relates particularly to the delivery of adult stem cells, which are extracted from cord blood or adults' bone marrow, blood, etc. and refers to primitive cells just before differentiation into cells of specific organs such as bone, liver, blood, etc. Adult stem cells include hematopoietic stem cells, mesenchymal stem cells, and neural stem cells, etc. that have attracted much attention as materials for regenerative medicine.

Cord blood (umbilical cord blood) contains large amounts of hematopoietic stem cells, and myeloid cells found in the marrow contain various types of stem cells including hematopoietic stem cells, which can produce blood and lymphocytes, mesenchymal stem cells, etc.

Adult stem cells are difficult to proliferate and have a strong tendency differentiate easily. Various types of adult stem cells can be used to regenerate organs and can differentiate to meet the characteristics of each organ after transplantation of stem cells.

Moreover, unlike embryonic stem cells extracted from human embryo, adult stem cells are extracted from body tissues that have already grown, such as bone marrow or brain cells, and thus can avoid the ethical controversy.

Technology related to fundamental and applied tissue engineering has been advanced for the purpose of developing transplantable artificial tissues as part of regenerative medicine. Specifically, studies including stem cell proliferation and differentiation, development of cytocompatible and biocompatible three-dimensional scaffolds, and construction of a variety of tissue engineering tools are now the most active research areas in regenerative medicine. Among them, three-dimensional scaffolds that are used to deliver stem cells or tissue cells therein are critical for the development of artificial tissues and organs.

Scaffold materials used for the regeneration of body tissues must act as a substrate or framework to which cells adhere to form three-dimensional tissues and must also function as a temporary barrier between transplanted cells and host cells, which means that the materials should have non-toxic biocompatibility such that they do not cause blood clotting and inflammatory response after transplantation. In addition, the scaffold materials must be biodegradable in vivo at a desired time when the transplanted cells have grown sufficiently to the point of being able to adequately function as a tissue. Therefore, the scaffolds are produced from synthetic or natural polymers or composites thereof and are manufactured into three-dimensional structures which have a variety of morphologies and properties. Synthetic biodegradable polymers that are currently being widely used include polyglycolic acid (PGA), polylactic acid (PLA), polylactic acid-polyglycolic acid copolymer (PLGA), poly-£-caprolactone (PCL), and derivatives and copolymers thereof. Naturally biodegradable polymers include collagen, alginate, hyaluronic acid, gelatin, chitosan, fibrin, etc. A variety of different forms of materials, such as sponges, gels, fibers, and microbeads, are applied for the fabrication of scaffolds, and the most popular ones are porous sponges and injectable hydrogels.

However, among various technical barriers in tissue engineering, the most critical one with regard to the scaffold is the development of a cytocompatible surface environment within the scaffold. In light of the fact that scaffolds are fundamental in providing a three-dimensional environment that is advantageous for cell adhesion and growth, the properties of the scaffold surface where cell adhesion occurs may play a critical role in determining the present and future behaviors of cells.

The polymer surface to which cells are directly attached is one of the most important factors in scaffold design. In general, cell adhesion to natural polymers is much easier to accomplish than that to synthetic polymers, and thus a method of surface coating or grafting of scaffolds with materials which cell surface receptors can recognize, such as fibronectin, an arginine-glycine-aspartic acid (RGD) peptide, vitronectin, laminin, etc., a method of treating a polymeric scaffold surface with a natural polymer, such as collagen, gelatin, fibrin, etc., or a method of preparing a hybrid scaffold from a mixture of synthetic and natural polymers has been conventionally used. However, these methods are only partly effective in ensuring cell adhesion and stem cell differentiation, and there limitations in creating a biomimetic surface environment that the cells recognize as their own environment.

Meanwhile, extracellular matrix (ECM) is a matrix that surrounds cells, occupies space between cells, and has a network structure composed mainly of proteins and polysaccharides. Components of the extracellular matrix include structural elements such as collagen and elastin; adhesive proteins such as fibronectin, laminin, vitronectin, and tenascin; proteoglycans such as chondroitin sulfates or heparan sulfates and core proteins; and hyaluronic acids consisting of polysaccharides only. These extracellular matrix components are the major substances that determine the shapes of tissues formed by aggregating cells, provide an environment where cells have normal functions, and are involved in cell differentiation.

Therefore many studies have been carried out, using the advantages of the extracellular matrix that provides an environment where cells have normal functions. For example, living tissues such as porcine small intestine submucosa (SIS), bladder, and skin, are directly decellularized to obtain a cell-free matrix, which is then used for cell transplantation. In addition, Korean Patent No. 10-715505 describes a method of preparing a cell-derived extracellular matrix scaffold. However the cell-derived extracellular matrix scaffold according to Korean Patent No. 10-715505 is useful only for cartilage regeneration.

Therefore, there is a need for developing a scaffold that provides a biomimetic environment that is useful even for regeneration of other tissue cells.

### SUMMARY OF THE INVENTION

An object of the present invention is to provide a stem cell carrier or scaffold which can deliver adult stem cells into the body and provide an environment where the adult stem cells can differentiate into bone tissue.

Moreover, the stem cell carrier transplanted into the body can withstand the weight and other mechanical load in a solid state and allows stem cells to be delivered into the body during transplantation. The stem cell carrier can be fixed and maintained in a transplanted area without undesired deformation or displacement, and after the transplantation of the carrier without affecting the adjacent anatomical and surgical structure, it does not require an additional graft or device to more firmly engage the surface of the carrier with the surface of adjacent issue.

Furthermore, the present invention provides a 3D bone scaffold that perfectly coincides with a bone defect using CAD/CAM, facilitating bone regeneration without any biological side effects.

In an embodiment of the present invention, the stem cell carrier is formed with a cubic structure having a plurality of through holes. That is, this cubic structure has a plurality of through-hole channels having a diameter of 400 to 500 micrometers formed with hydroxyapatite (HA) or hybrid materials thereof and thus provides a surface and a scaffold where stem cells can be cultured.

Moreover, in order to form a stem cell scaffold for bone regeneration, the bone defect is diagnosed and imaged using CT and MRI, and the image information is sent to a CAD/CAM to form the scaffold in a three-dimensional manner using the cubic structure.

Adult stem cells are stored in the formed scaffold of the carrier, which is then fixed to the bone defect.

### BRIEF DESCRIPTION OF THE DRAWINGS

The above and other features and advantages of the present invention will become more apparent by describing in detail exemplary embodiments thereof with reference to the attached drawings in which:

FIG. 1 is a perspective view showing a stem cell carrier with a cubic structure;

FIG. 2 is a plan view of FIG. 1; and

FIG. 3 is an enlarged perspective view of a single channel forming a part of FIG. 1.

### DETAILED DESCRIPTION OF THE INVENTION

Hereinafter, the present invention will be described in detail with reference to the accompanying drawings.

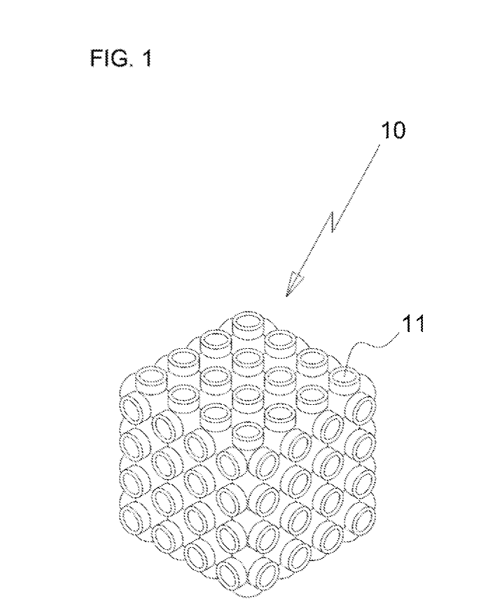

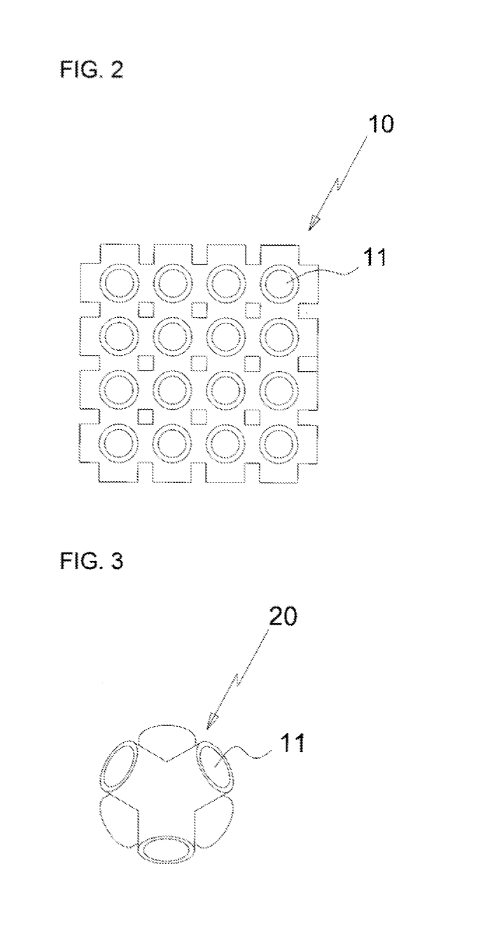

As shown in FIGS. 1 and 2, a stem cell carrier 10 has a cubic structure comprising a plurality of through holes 11 formed to penetrate through the cubic structure.

The stem cell carrier 10 is composed of a single body which may be divided by physical force into single units 20 as shown in FIG. 3.

The stem cell carrier 10 is composed of hydroxyapatite (HA), β-TCP (tricalcium phosphate), or mixtures thereof, and these materials the main components that constitute bones and teeth of the body and have the advantages that they are more flexible than simple ceramic, have good biocompatibility, and do not cause deterioration and unnecessary biological reactions.

The roles of the stem cell carrier 10 are to activate cells in the body to secrete a new matrix, thus regenerating differentiated tissue. Stem cells supported on a scaffold that is non-toxic to the body and is completely biodegradable, and the hydroxyapatite (HA) and β-TCP have sufficient effect as tissue engineering porous scaffolds.

A method for bone regeneration using the stem cell carrier 10 configured in the above-described manner will be described below.

First, a bone defect is imaged using CT and MRI, and the image information is sent to a CAD/CAM to form a scaffold using the stem cell carrier 10 prepared to coincide with the bone defect.

Stem cells are stored in the formed scaffold, and the scaffold is put on the bone defect and then fixed using a fixing means composed of a biocompatible material such as PLA.

As described above, according to the stem cell carrier of the present invention, the treatment of bone damage can reduce the risks of immune rejection and infection in patients and allows stem cells isolated from the body to be cultured and differentiated, thus maintaining homeostasis in the defect tissue.

1: A stem cell carrier comprising a plurality of single units, the plurality of single units formed three-dimensionally interconnected to each other to form a cubic structure, the plurality of single units made of a porous material, wherein each single unit has three-dimensionally formed through holes crossing each other, each through hole having 400 to 500 micrometers in diameter, and the through holes of the plurality of single units are interconnected to each other to form a plurality of channels which are formed from one side of the cubic structure to the other side.

2: The stem cell carrier of claim 1, wherein the porous material is selected from the group consisting of hydroxyapatite (HA), β-TCP (tricalcium phosphate), and mixtures thereof.

3: A method for bone regeneration with 3D customized computer-aided design and computer-aided manufacturing (CAD/CAM), the method comprising:
imaging a bone defect using CT and MRI;
sending the image information of the bone defect to a CAD/CAM;
forming a scaffold by shaping the stem cell carrier of claim 1 to have the bone defect;
storing stem cells in the formed scaffold; and
fixing the scaffold storing the stem cells with a fixing device comprised of a biocompatible material.

4: The stem cell carrier of claim 1, wherein each single unit has a first through hole, a second through hole, a third through hole which perpendicularly cross each other; and
the plurality of channels comprise first channels formed of interconnected first through holes of the plurality of single units, second channels formed of interconnected second through holes of the plurality of single units, and third channels formed of interconnected third through holes of the plurality of single units.

5: The method of claim 3, wherein each single unit has a first through hole, a second through hole, a third through hole which perpendicularly cross each other; and
the plurality of channels comprise first channels formed of interconnected first through holes of the plurality of single units, second channels formed of interconnected second through holes of the plurality of single units, and third channels formed of interconnected third through holes of the plurality of single units.
